SEQUENCE LISTING (2 sequences)

SEQ ID NO: 1 - protein, 18 aa, Artificial Sequence
  Description of Artificial Sequence Synthetic peptide
CPGQEREGTPPIEERKVE

SEQ ID NO: 2 - protein, 102 aa, Homo sapiens
MSARVRSRSRGRGDGQEAPDVVAFVAPGESQQEEPPTDNQDIEPGQEREGTPPIEERKVEGDCQEMDLEKTRSERGDGSDVKEKTPPNPKHAKTKEAGDGQP

(19) United States
(12) Patent Application Publication
(10) Pub. No.: US 2005/0287567 A1
(43) Pub. Date: Dec. 29, 2005

(54) JM-27 as a marker of benign prostatic hyperplasia

(75) Inventor: Robert H. Getzenberg, Lutherville, MD (US)

Correspondence Address: FOLEY AND LARDNER LLP;SUITE 500, 3000 K STREET NW, WASHINGTON, DC, 20007, US

(73) Assignee: University of Pittsburgh

(21) Appl. No.: 11/133,432

(22) Filed: May 20, 2005

Related U.S. Application Data
(60) Provisional application No. 60/580,392, filed on Jun. 18, 2004.

(51) Int. Cl. C12Q 1/68; G01N 33/53; G01N 33/537; G01N 33/543

(52) U.S. Cl. 435/6; 435/7.92

(57) ABSTRACT

A serum assay for JM-27 identifies individuals having benign prostate hyperplasia (BPH). By means of the serum assay, methods of diagnosing BPH, methods of determining the severity of BPH, methods of monitoring the course of BPH therapy and methods of monitoring the progression of BPH are performed.

### STATEMENT REGARDING GOVERNMENT SPONSORED RESEARCH

This invention was made with support from the U.S. Federal Government under grant No. DK-063593. Thus, the U.S. Federal Government may have certain rights to this invention.

### FIELD OF THE INVENTION

This invention relates to methods of detecting JM-27 in the serum of individuals with benign prostatic hyperplasia (BPH). More particularly, the invention relates to methods that employ an anti-JM-27 antibody, in connection with immunoassays, to detect the presence of JM-27 in serum samples. The methods are useful for diagnosing BPH and determining the severity of BPH, as well as for monitoring the effectiveness of BPH therapy.

### BACKGROUND OF THE INVENTION

Abnormalities of prostate growth, including cancer and benign prostatic hyperplasia (BPH), produce some of the most common, costly, and devastating diseases occurring in men. BPH constitutes the most common benign tumor in men over the age of 60. In fact, it is estimated that one in four men living to the age of 80 will require treatment for this disease. BPH usually is noted clinically-after the age of 50, and the incidence of BPH increases with age. As many as two thirds of men between the ages of 40 and 49, however, demonstrate histological evidence of the disease.

The anatomic location of the prostate at the bladder neck enveloping the urethra plays an important role in the pathology of BPH, including bladder outlet obstruction. Two prostate components are thought to play a role in bladder outlet obstruction. The first is the relative increase in prostate tissue mass. The second component is the prostatic smooth muscle tone.

The causative factors of BPH in men have been intensively studied. See Ziada et al., Urology 53: 1-6. (1999). In general, the two most important factors appear to be aging and the presence of functional testes. Although these factors appear to be key to the development of BPH, both are nonspecific.

BPH exists in both symptomatic and asymptomatic forms, making it particularly difficult to diagnose. Gene expression profiling studies using DNA microarrays have identified JM-27 as a relatively prostate specific protein that is associated with BPH. See Prakash et al., Proc. Nat'l Acad. Sci. USA 99: 7598-03 (2002), and PCT applications WO 03/008561 and WO 02/10338. Such studies have shown that JM-27 is significantly upregulated in symptomatic BPH as compared to histologic BPH.

It is desirable to identify individuals with BPH, particularly severe BPH, in order to apply therapeutic approaches while they may be more effective and before any bladder damage occurs. However, BPH is often difficult to decipher in tissue biopsies, and it is currently difficult to guage the severity of BPH using vague symptomatic scoring.

It is also desirable to differentiate between individuals with elevated prostate-specific antigen (PSA) levels due to BPH, and individuals with elevated PSA levels due to prostate cancer. Current methods of diagnosing prostate cancer rely heavily on detecting elevated PSA levels, which may actually be the result of BPH. The ability to differentiate between the causes of elevated PSA would reduce the number of unnecessary biopsies in BPH patients.

There remains a need for an efficient and simple method of identifying individuals that suffer from BPH and of determining the severity of BPH in an individual. Similarly, there remains a need for an efficient and simple method of differentiating individuals with BPH from individuals with prostate cancer.

There also remains a need for a method of monitoring the course of BPH therapy, and tools that would inform decisions about BPH therapy.

### SUMMARY OF THE INVENTION

The inventors have discovered that JM-27 is detectable in the serum of individuals having BPH, and that the level of JM-27 detected in the serum correlates to the severity of BPH. Specifically, the serum level of JM-27 in symptomatic BPH is less than the serum level of JM-27 in asymptomatic BPH. The inventors also have discovered that JM-27 serum levels are useful for monitoring the course of BPH therapy.

In accord with these discoveries, the invention provides a serum-based assay for JM-27. The assay includes the steps of (a) providing a serum sample, and (b) detecting JM-27 in the serum sample. The detection method of step (b) may employ any known protein detection method, including ELISA, immunoprecipitation and western blotting.

In another aspect, the invention provides a method of diagnosing BPH that comprises (a) providing a serum sample from an individual, (b) detecting the level of JM-27 present in the serum sample, and (c) correlating the detected level of JM-27 to a diagnosis of BPH. The detection method of step (b) may employ any known protein detection method, including ELISA, immunoprecipitation and western blotting. A preferred embodiment employs ELISA. In one embodiment, the JM-27 serum-based assay comprises the steps of (i) placing an anti-JM-27 antibody in contact with a solid phase coated with serum, (ii) removing unbound anti-JM-27 antibody; (iii) contacting a secondary antibody with the solid phase, (iv) removing unbound secondary antibody; (v) detecting the level of secondary antibody that is bound to the solid phase; wherein the level of (v) indicates the level of JM-27 in the serum.

In particular embodiments, the diagnostic methods are useful for differentiating between individuals having symptomatic BPH, asymptomatic BPH, and prostate cancer.

The invention also provides a method of determining the severity of BPH, and stratifying patients that will most benefit from BPH therapy. This method comprises (a) providing a serum sample from an individual, (b) detecting the level of JM-27 present in the serum sample, and (c) correlating the detected level of JM-27 to the severity of BPH. Patients with low serum levels of BPH, indicating a more severe disease condition, may be selected for BPH therapy.

The invention also provides a method of monitoring the effectiveness of BPH therapy or the course of BPH progression in an individual. This method comprises (a) providing a serum sample from an individual, (b) detecting the level of JM-27 present in the serum sample, and (c) comparing the detected level of JM-27 to an earlier serum level of JM-27 in the same individual. An increase in the serum JM-27 level over time indicates that BPH has improved, whereas a decrease in the serum JM-27 level over time indicates that BPH has worsened.

In a particular embodiment, the method of monitoring the effectiveness of BPH therapy is used to monitor the effectiveness of androgen therapy, such as therapy with a 5-α-reductase inhibitor. The method is particularly useful for this purpose. Androgens themselves are not specific markers of BPH, but JM-27, which is androgen-regulated, is a specific marker of BPH.

For each above-described embodiment of the invention, an anti-JM-27 antibody directed specifically against a JM-27 protein, natural variant or identifying fragment may be employed. In particular, antibodies directed specifically against a JM-27 protein fragment having an amino acid sequence represented by SEQ ID NO: 1 may be employed. Both monoclonal antibodies and polyclonal antibodies directed against JM-27 constitute useful reagents. In particular embodiments, a secondary antibody, such as a reporter molecule labeled antibody, is employed to detect the amount of JM-27 in a serum sample. The reporter molecule labeled antibody used in the assay may be an enzyme that allows for quantification of the secondary antibody in the sample.

### BRIEF DESCRIPTION OF THE FIGURES

FIG. 1 depicts the amino acid sequence (SEQ ID NO: 2) for JM-27.

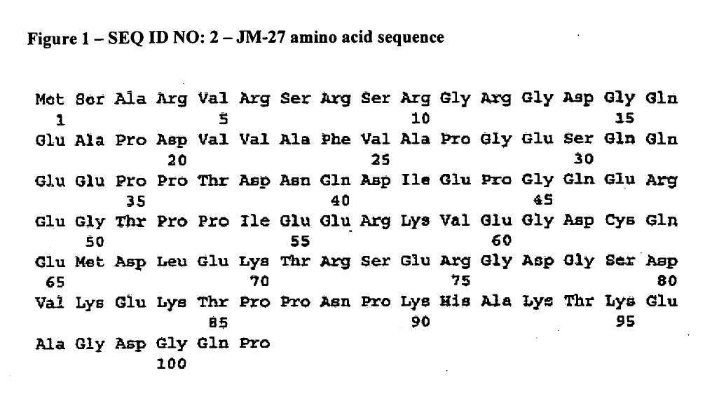

FIG. 2 depicts the correlation of serum JM-27 level to the severity of symptoms in BPH patients. The figure also shows that prostate cancer does not effect JM-27 levels.

FIG. 3 depicts the sensitivity of the JM-27 ELISA assay.

FIG. 4 further depicts the correlation of serum JM-27 level to the severity of symptoms in BPH patients. The figure also shows that prostate cancer does not effect JM-27 levels.

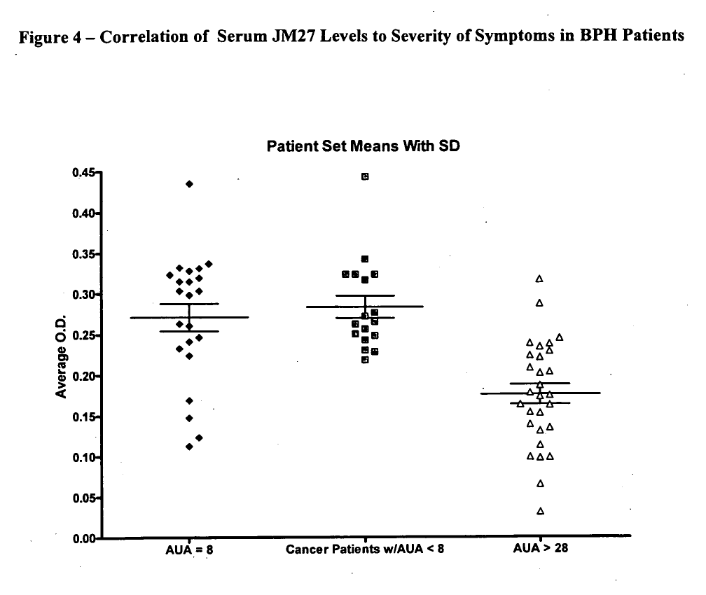

### DETAILED DESCRIPTION OF THE PREFERRED EMBODIMENTS

### I. DEFINITIONS

For convenience, the meaning of certain terms and phrases employed in the specification, examples, and appended claims are provided below.

The singular forms “a,” “an,” and “the” include plural reference unless the context clearly dictates otherwise.

The term “antibody” includes any synthetic or genetically engineered protein that is functionally capable of binding an epitopic determinant of JM-27. It also refers to a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment.

An “antibody fragment” is a portion of an antibody such as F(ab′)₂, F(ab)₂, Fab′, Fab, Fv, scFv (single chain Fv) and the like. Regardless of structure, an antibody fragment binds with the same JM-27 antigen that is recognized by the intact antibody.

The term “antibody fragment” also includes any synthetic or genetically engineered protein that acts like an antibody by binding to a specific JM-27 antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region. The Fv fragments may be constructed in different ways as to yield multivalent and/or multispecific binding forms. In the former case of multivalent, they react with more than one binding site against the specific epitope, whereas with multispecific forms, more than one epitope (either of the antigen or even against the specific antigen and a different antigen) is bound.

In this description, the term “antibody component” includes both an entire antibody, a fusion protein, and fragments of any of them.

A “chimeric antibody” is a recombinant protein that contains the variable domains of both the heavy and light antibody chains, including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A “humanized antibody” is a recombinant protein in which the CDRs from an antibody from one species, e.g., a rodent antibody, are transferred from the heavy and light variable chains of the rodent antibody into human heavy and light variable domains. The constant domains of the antibody molecule are derived from those of a human antibody.

A “human antibody” is an antibody obtained from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7: 13 (1994), Lonberg et al., Nature 368: 856 (1994), and Taylor et al., Int. Immun. 6: 579 (1994). A fully human antibody also can be constructed by genetic or chromosomal transfection methods, as well as phage display technology, all of which are known in the art. See for example, McCafferty et al., Nature 348: 552-553 (1990), for the production of human antibodies and fragments thereof in vitro, from immunoglobulin variable domain gene repertoires from unimmunized donors. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats. For a review, see Johnson and Chiswell, Current Opinion in Structural Biol. 3: 5564-571 (1993).

“Benign Prostate Hyperplasia” refers to a non-malignant enlargement of the prostate, a frequent occurrence in aging men. The symptoms of BPH vary, but the most common ones involve changes or problems with urination, such as (a) a hesitant, interrupted, weak stream, (b) urgency and leaking or dribbling, and (c) more frequent urination, especially at night. The disease also contributes significantly to what is termed lower urinary tract symptoms (LUTS).

Two distinct types of BPH exist: (a) asymptomatic, or histologic, and (b) symptomatic, which includes severe BPH conditions. In the former, one or more BPH lesions exist within the prostate, but the patient does not experience any symptoms. In the latter, a patient experiences a range of symptoms. Patients most severely affected with symptomatic BPH may require surgical intervention.

“Biomolecular sequence” or “sequence” refers to all or a portion of a polynucleotide or polypeptide sequence.

“Diagnosis” and “diagnosing” generally includes a determination of a subject's susceptibility to a disease or disorder, a determination as to whether a subject is presently affected by a disease or disorder, a prognosis of a subject affected by a disease or disorder, and therametrics (e.g., monitoring a subject's condition to provide information as to the effect or efficacy of therapy).

“Epitope” denotes any determinant capable of specific interaction with the antibodies of the invention. Epitopic determinants usually comprise chemically active surface groupings of molecules such as amino acids or sugar side chains and usually have specific three dimensional structural characteristics, as well as specific charge characteristics.

A “fragment of a protein” refers to a protein that is a portion of another protein. For example, fragments of proteins may comprise polypeptides obtained by digesting full-length protein isolated from cultured cells. In one embodiment, a protein fragment comprises at least about 6 amino acids. In another embodiment, the fragment comprises at least about 10 amino acids. In yet another embodiment, the protein fragment comprises at least about 16 amino acids.

“Hyperplasia” refers to cells that exhibit abnormal multiplication or abnormal arrangement in a tissue. Included in the term “hyperplasia,” are benign cellular proliferative disorders, including benign tumors.

“Individual,” “subject,” “host,” and “patient,” used interchangeably herein, refer to any mammalian subject for whom diagnosis, treatment, or therapy is desired. In one preferred embodiment, the individual, subject, host, or patient is a human. Other subjects may include, but are not limited to, cattle, horses, dogs, cats, guinea pigs, rabbits, rats, primates, and mice.

“Isolated” refers to a polynucleotide, a polypeptide, an immunoglobulin, or a host cell that is in an environment different from that in which the polynucleotide, the polypeptide, the immunoglobulin, or the host cell naturally occurs.

“JM-27” refers to the protein represented by SEQ ID NO: 2. The protein is expressed only in the prostate and certain female reproductive tissues, e.g., uterine tissue. JM-27 transcripts are up-regulated 17-fold in symptomatic BPH; JM-27 protein expression also is upregulated in BPH. JM-27 is homologous to a family of MAGE/GAGE-like proteins containing RGD motifs frequently found in cell adhesion proteins. Homologues, natural variants, and fragments of JM-27 that retain the ability to function as markers of BPH are within the scope of this invention.

“Label” refers to agents that are capable of providing a detectable signal, either directly or through interaction with one or more additional members of a signal producing system. Labels that are directly detectable and may find use in the invention include fluorescent labels. Specific fluorophores include fluorescein, rhodamine, BODIPY, cyanine dyes and the like. The invention also contemplates the use of radioactive isotopes, such as ³⁵S, ³²P, ³H, and the like as labels. Colorimetric labels such as colloidal gold or colored glass or plastic (e.g., polystyrene, polypropylene, latex) beads may also be utilized. Additionally, catalytic enzymes may function as labels.

“Modulate” refers to increasing or decreasing an indicated phenomenon. A JM-27 modulator increases or decreases one or more activities of JM-27, either directly or indirectly. For example, a JM-27 modulator may directly impact protein activity by binding to JM-27, and may indirectly impact protein activity by affecting the transcription, translation or post-translational modification of JM-27.

“Neoplasia” refers to abnormal new growth, which results in a tumor. Unlike hyperplasia, neoplastic proliferation persists even in the absence of the original stimulus and is characterized as uncontrolled and progressive. Malignant neoplasms, or malignant tumors, are distinguished from benign tumors in that the former show a greater degree of anaplasia and have the properties of invasion and metastasis.

“Polypeptide” and “protein,” used interchangeably herein, refer to a polymeric form of amino acids of any length, which may include translated, untranslated, chemically modified, biochemically modified, and derivatized amino acids. A polypeptide or protein may be naturally occurring, recombinant, or synthetic, or any combination of these. Moreover, a polypeptide or protein may comprise a fragment of a naturally occurring protein or peptide. A polypeptide or protein may be a single molecule or may be a multi-molecular complex. In addition, such polypeptides or proteins may have modified peptide backbones. The terms include fusion proteins, including fusion proteins with a heterologous amino acid sequence, fusions with heterologous and homologous leader sequences, with or without N-terminal methionine residues, immunologically tagged proteins, and the like.

“Sequence Identity” refers to a degree of similarity or complementarity. There may be partial identity or complete identity. A partially complementary sequence is one that at least partially inhibits an identical sequence from hybridizing to a target polynucleotide; it is referred to using the functional term “substantially identical” The inhibition of hybridization of the completely complementary sequence to the target sequence may be examined using a hybridization assay (Southern or Northern blot, solution hybridization and the like) under conditions of low stringency. A substantially identical sequence or probe will compete for and inhibit the binding (i.e., the hybridization) of a completely identical sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding, the probe will not hybridize to the second non-complementary target sequence.

Another way of viewing sequence identity in the context of two nucleic acid or polypeptide sequences includes reference to residues in the two sequences that are the same when aligned for maximum correspondence over a specified region. As used herein, percentage of sequence identity means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

“Serum sample” refers to a specimen that comprises the components of serum. Exemplary serum samples are whole blood, plasma and serum. Preferred serum samples are plasma and serum.

The terms “treatment,” “treating,” “treat,” and the like refer to obtaining a desired pharmacological and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

### II. SERUM ASSAYS FOR DETECTING JM-27

In one aspect, the invention provides an assay for detecting JM-27, comprising the steps of (a) providing a serum sample from an individual, and (b) detecting the level of JM-27 in said serum sample. Any of the numerous protein detection methods well-known in the art may be used for the detection step, including immunoassay, enzyme-linked immunosorbent assay (ELISA), radioimmunoassay (RIA), western blotting, and functional assays for JM-27.

Several methods for detecting the presence of and/or measuring a level of JM-27 polypeptide in a serum sample employ an antibody specific for JM-27. Specifically, the method for detecting the presence of JM-27 polypeptides in a serum sample may comprise the step of contacting the sample with an antibody and detecting the binding of the antibody with the JM-27 in the sample. More specifically, the antibody may be labeled so as to produce a detectable signal using compounds including, but not limited to, a radiolabel, an enzyme, a chromophore and a fluorophore.

For example, an immunofluorescence assay may be easily performed on serum without first isolating the JM-27 polypeptide. Serum proteins are first fixed onto a solid support, such as a microscope slide or microtiter well. Next, the fixed proteins are exposed to an antibody specific for the JM-27 polypeptide. To increase the sensitivity of the assay, the fixed proteins may be further exposed to a second antibody, which is labeled and binds to the first antibody, which is specific for the JM-27 polypeptide. Typically, the secondary antibody is detectably labeled, e.g., with a fluorescent marker. The JM-27 polypeptide will be fluorescently labeled and easily visualized.

As will be readily apparent to the ordinarily skilled artisan upon reading the present specification, the detection methods and other methods described herein may be readily varied. Such variations are within the intended scope of the invention. For example, in the above detection scheme, the antibody for use in detection may be immobilized on a solid support, and the serum sample contacted with the immobilized probe. Binding of JM-27 in the serum sample to the antibody may then be detected in a variety of ways.

Detection of specific binding of an antibody specific for JM-27, when compared to a suitable control, is an indication that JM-27 polypeptides are present in the sample. Suitable controls include a sample known not to contain JM-27 polypeptides and a sample contacted with an antibody not specific for the encoded polypeptide, e.g., an anti-idiotype antibody. A variety of methods to detect specific antibody-antigen interactions are known in the art and may be used in the method, including, but not limited to, standard immunohistological methods, immunoprecipitation, an enzyme immunoassay, and a radioimmunoassay. In general, the specific antibody will be detectably labeled, either directly or indirectly. Direct labels include radioisotopes; enzymes whose products are detectable (e.g., luciferase, 3-galactosidase, and the like); fluorescent labels (e.g., fluorescein isothiocyanate, rhodamine, phycoerythrin, and the like); fluorescence emitting metals (e.g., 112Eu, or others of the lanthanide series, attached to the antibody through metal chelating groups such as EDTA); chemiluminescent compounds (e.g., luminol, isoluminol, acridinium salts, and the like); bioluminescent compounds (e.g., luciferin, aequorin (green fluorescent protein), and the like). The antibody may be attached (coupled) to an insoluble support, such as a polystyrene plate or a bead. Indirect labels include second antibodies specific for antibodies specific for the encoded polypeptide (“first specific antibody”), wherein the second antibody is labeled as described above; and members of specific binding pairs, e.g., biotin-avidin, and the like. The serum sample may be brought into contact with and immobilized on a solid support or carrier, such as nitrocellulose, that is capable of immobilizing cells, cell particles, or soluble proteins. The support may then be washed with suitable buffers, followed by contacting with a detectably-labeled first specific antibody. Detection methods are known in the art and will be chosen as appropriate to the signal emitted by the detectable label. Detection is generally accomplished in comparison to suitable controls and to appropriate standards.

In a particular embodiment, an assay for detecting JM-27 comprises (i) placing an anti-JM-27 antibody in contact with a solid phase coated with serum, (ii) removing unbound anti-JM-27 antibody; (iii) contacting a secondary antibody with the solid phase, (iv) removing unbound secondary antibody; (v) detecting the level of secondary antibody that is bound to the solid phase, where the level of (v) indicates the level of JM-27 in the serum.

### III. METHODS OF DIAGNOSING BPH

In another aspect, the invention provides a method of diagnosing BPH, comprising (a) providing a serum sample from an individual, (b) detecting the level of JM-27 present in the serum sample, and (c) correlating the detected level of JM-27 to a diagnosis of BPH.

The method may comprise (a) determining the level of JM-27 in a serum sample obtained from a patient, (b) comparing the level of JM-27 in the serum sample to the level of JM-27 in a normal biological sample, and (c) correlating the level of JM-27 to a diagnosis of BPH.

A related aspect of the invention provides a method for determining a patient's predisposition to BPH. The method entails (a) determining the level of JM-27 in a serum sample obtained from a patient, (b) comparing the level of JM-27 in said serum sample to the level of JM-27 in a normal biological sample, and (c) correlating the level of JM-27 to a diagnosis of a predisposition to BPH.

Specifically, in diagnosing a patient, the level of JM-27 in a serum sample obtained from a patient may be determined. Next, the level of JM-27 in the patient's serum sample may be compared to the JM-27 level from a normal serum sample or to a standard curve of serum JM-27 levels and correlated to a positive or negative diagnosis of BPH. A patient's predisposition to BPH may be determined using a similar method.

In one embodiment, the severity of BPH may be determined by comparing patient JM-27 polypeptide levels to normal JM-27 polypeptide levels or to a standard curve of serum JM-27 polypeptide levels. Lower levels of JM-27 correlate to more severe, or advanced, BPH. Thus, the serum level of JM-27 may be used to discriminate between symptomatic and asymptomatic BPH.

In another embodiment, BPH and prostate cancer can be distinguished by comparing patient JM-27 polypeptide levels to normal JM-27 polypeptide levels. Unlike BPH, prostate cancer does not significantly influence serum levels of JM-27. As noted above, current methods of diagnosing prostate cancer rely on detecting elevated PSA levels, which may actually result from BPH. The ability to definitively diagnose BPH, and thereby differentiate between the causes of elevated PSA will reduce the number of unnecessary biopsies in BPH patients.

### IV. METHODS OF MONITORING BPH THERAPY

Serum levels of JM-27 are of particular interest as markers that will detect changes in BPH progression and/or monitor the efficacy of various therapies and preventive interventions. For example, the serum level of JM-27 can be indicative of advanced disease, and therefore warrant more aggressive therapy for a patient or vice versa. Thus, JM-27 serum assays of the invention also are useful for assessing BPH therapy and making decisions about managing BPH in an individual. Determining the serum level of JM-27 in a patient and comparing a patient's profile with known expression in normal tissue and variants of the disease allows a determination of the best possible treatment for a patient, both in terms of specificity of treatment and in terms of comfort level of the patient.

In one embodiment, such methods comprises (a) providing a serum sample from an individual, (b) detecting the level of JM-27 present in the serum sample, and (c) comparing the detected level of JM-27 to an earlier serum level of JM-27 in the same patient. Preferably, the therapy being evaluated comprises an androgen therapy, such as the administration of doxazosin or finasteride.

### V. ANTIBODIES AND THEIR USE WITH THE INVENTION

Polyclonal and monoclonal antibodies of the invention are immunoreactive with JM-27 or immunogenic fragments of JM-27. If desired, polyclonal antibodies can be further purified, for example, by binding to and elution from a matrix to which JM-27 polypeptide is bound or by utilizing non-JM-27 proteins, preferably including NMPs, to selectively remove non-specific antibodies. Antibodies which consist essentially of pooled monoclonal antibodies with different epitopic specificities, as well as distinct monoclonal antibody preparations, are provided.

Anti-JM-27 antibodies for use with the invention may be readily prepared by one skilled in the art. As preferred example, the inventors have prepared a monoclonal antibody from a clone designated 9G4.2. The sensitivity and specificity of this antibody in serum JM-27 assays described in the examples below are 85% and 76%, respectively.

An identifying peptide fragment of JM-27 can be used to prepare antibodies specific for JM-27. Antibodies can be prepared that bind a distinct epitope of the peptide fragment or can recognize an epitope created by a combination of peptide fragments, either in overlapping regions or to secondary structure elements of, for example dimers or trimers of peptide fragments.

Conventional methods can be used to prepare the antibodies. For example, by using a JM-27 peptide, polyclonal antisera or monoclonal antibodies can be made using standard methods. This invention also contemplates chimeric antibody molecules, made by methods known to those skilled in the art.

The antibodies may be labeled with a detectable marker including various enzymes, fluorescent materials, luminescent materials and radioactive materials that are known to those skilled in the art.

Antibodies reactive against naturally occurring JM-27 and fragments thereof may be used to detect JM-27, including the peptide sequence in serum samples. For example, such antibodies may be used in any known immunoassay and immunological method that relies on the binding interaction between an antigenic determinant of the JM-27 protein and the antibodies. Examples of such assays are radioimmunoassays, Western immunoblotting, enzyme immunoassays (e.g. ELISA), immunofluorescence, immunoprecipitation, latex agglutination, and immunohistochemical tests. Thus, the antibodies may be used to identify or quantify the amount of JM-27 in a sample and thus, may be used as a diagnostic indicator of BPH.

A sample may be tested for the presence or absence of JM-27 by contacting the sample with an antibody specific for an epitope of the peptide fragment, which antibody is capable of being detected after it becomes bound to JM-27 in the sample, and assaying for antibody bound to JM-27 in the sample, or unreacted antibody.

In the method of the immunoassay, a predetermined amount of a sample or concentrated sample is mixed with antibody or labeled antibody. The amount of antibody used in the method is dependent upon the labeling agent chosen. The amount of JM-27 bound to the antibody or labeled antibody may then be detected by methods known to those skilled in the art. The sample or antibody may be insolubilized, for example, the sample or antibody can be reacted using known methods with a suitable carrier. Examples of suitable carriers are Sepharose or agarose beads. When an insolubilized sample or antibody is used, JM-27 bound to antibody or unreacted antibody is isolated by washing. For example, when the sample is blotted onto a nitrocellulose membrane, the antibody bound to JM-27 is separated from the unreacted antibody by washing with a buffer, for example, phosphate buffered saline (PBS) with bovine serum albumin (BSA).

When labelled antibody is used, the presence of JM-27 can be determined by measuring the amount of labelled antibody bound in the sample. The appropriate method of measuring the labelled material is dependent upon the labelling agent.

When unlabelled antibody is used in a method of the invention, the presence of JM-27 can be determined by measuring the amount of antibody bound to one or more JM-27 molecules using substances that interact specifically with the antibody to cause agglutination or precipitation. In particular, labelled antibody against an antibody specific for JM-27, can be added to the reaction mixture. The antibody against an antibody specific for a peptide of the invention can be prepared and labelled by conventional procedures known in the art which have been described herein. The antibody against an antibody specific for a JM-27 may be a species specific anti-immunoglobulin antibody or monoclonal antibody, for example, goat anti-rabbit antibody may be used to detect rabbit antibody specific for a peptide of the invention.

A preferred JM-27 fragment for generating antibodies is a peptide having the following sequence CPGQEREGTPPIEERKVE (SEQ ID NO: 1). Minor modifications of JM-27 primary amino acid sequences also may result in useful peptides. Such minor modifications include “conservative substitutions.” As used herein, conservative substitution denotes the replacement of an amino acid residue by a structurally similar residue. Examples of conservative substitutions include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like.

JM-27 and fragments thereof can be synthesized by the well known solid phase peptide synthesis methods described, for example, by Merrifield, J. Am. Chem. Soc. 85: 2149 (1962), and by Stewart and Young, SOLID PHASE PEPTIDES SYNTHESIS 27-62 (Freeman Publ., 1969).

A preferred method for the identification and isolation of antibody binding domains which exhibit binding with JM-27 is the bacteriophage X vector system. This vector system has been used to express a combinatorial library of Fab fragments from the mouse antibody repertoire in Escherichia coli, see Huse et al., Science 246: 1275-81 (1989), and from the human antibody repertoire. Nullinax et al., Proc. Nat'l Acad. Sci. USA 87: 8095-99 (1990).

Antibodies of the invention are suitable for use in immunoassays in which they can be utilized in liquid phase or bound to a solid phase carrier. In addition, the antibodies in these immunoassays can be detectably labeled in various ways. Examples of types of immunoassays that can utilize antibodies of the invention are competitive and non-competitive immunoassays in either a direct or indirect format. Examples of such immunoassays are the radioimmunoassay (RIA) and the sandwich (immunometric) assay. Detection of the antigens using the antibodies of the invention can be performed utilizing immunoassays that are run in either the forward, reverse, or simultaneous modes, including immunohistochemical assays on physiological samples. Alternatively, the antibody of the invention can be used to detect JM-27 present in electrophoretically dispersed gel protocols such as Western blots and two-dimensional gels.

Antibodies of the invention can be bound to many different carriers and used to detect the presence of JM-27. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention.

In performing the assays it may be desirable to include certain “blockers” in the incubation medium (usually added with the labeled soluble antibody). The “blockers” are added to ensure that non-specific proteins, proteases, or anti-heterophilic immunoglobulins to anti-JM-27 immunoglobulins present in the experimental sample do not cross-link or destroy the antibodies on the solid phase support, or the radiolabeled indicator antibody, to yield false positive or false negative results. The selection of “blockers” therefore may add substantially to the specificity of the assays described in the present invention.

It has been found that a number of nonrelevant (i.e., nonspecific) antibodies of the same class or subclass (isotype) as those used in the assays (e.g., IgG1, IgG2a, IgM, etc.) can be used as “blockers.” The “blockers” are used at a level high enough to maintain the proper sensitivity yet inhibit any unwanted interference by mutually occurring cross reactive proteins in the specimen (normally, 1-100 μg/μl).

### VI. EXAMPLES

The following non-limiting examples further illustrate certain aspects of the invention in detail. These examples are not to be construed as limiting the invention's scope.

### Example 1

### Peptide Synthesis and Antibody Production

An anti-peptide antibody against the JM-27 protein was generated against a synthetic peptide encoded for in the sequence of the JM-27 gene. A synthetic peptide, CPGQEREGTPPIEERKVE (SEQ ID NO: 1), which is amino acid residues 44-60 of human JM-27, was used to prepare polyclonal antibodies in rabbits. This peptide may also be used to prepare additional monoclonal and polyclonal antibodies against the JM-27 protein.

### Example 2

### Immunohistochemistry Analysis

Immunohistochemistry tests were performed on formalin-fixed paraffin-embedded sections of tissue obtained from various prostate tissue samples. The immunohistochemical examination was performed with the standard avidin-biotin technique, and a protease pretreatment step was included. Group 1 included normal prostate tissue from individuals age 20 and younger. Group 2 included prostate tissue from individuals between the ages of 20 and 50. Group 3 consisted of prostate tissue from individuals with symptomatic benign prostatic hyperplasia. Group 4 consisted of prostate tissue from individuals older than 50 years of age. Compared with normal prostate or asymptomatic BPH, JM-27 was found to be overexpressed and upregulated only in symptomatic BPH.

### Example 3

### Microarray Analysis

Microarray experiments run from more than 570 samples and covering a broad range of normal human tissue showed that JM-27 is expressed only in the prostate (and certain other female reproductive tissues). As such, JM-27 may be used as a marker for BPH as well as a therapeutic target for BPH.

Other microarray studies using prostate specific samples from different groups of patients revealed that JM-27 is upregulated by as much as seventeen-fold in symptomatic BPH patients when compared to normal and asymptomatic BPH patients with a neoplastic prostate. The prostate samples that were studied were from (1) individuals age 20 and younger with normal prostate tissue; (2) individuals between the ages of 20 and 50; (3) individuals with symptomatic BPH; and (4) individuals over the age of 50.

### Example 4

### Serum-Based ELISA Assay

An ELISA assay was developed to detect JM-27 in the serum of individuals with BPH. The preferred format is an indirect immunoassay in which the serum proteins are bound to a microtiter plate. The bound JM-27 protein was detected with an anti-JM-27 antibody, and subsequently detected with a secondary antibody. The ELISA assay can detect differences in levels of JM-27 in the serum, which can then be correlated with the type and severity of BPH.

To perform the ELISA, 100 ul of each specimen is plated per microtiter plate well. The plates are then incubated overnight at room temperature with shaking. Blocking is then performed with 1% milk/BSA in TBS with Tween-20, followed by an incubation performed with 2.5% milk/BSA in TBS with Tween-20. The plates are then washed three times and blocked with blocking buffer (1% milk or BSA) and incubated at room temperature for 30 minutes with shaking. The JM-27 antibody is diluted in antibody dilution buffer (2.5% milk or BSA) to a final concentration of 1:50 and 100 ul of the solution is added to each test well. The JM-27 antibody is not added to blank wells or to IgG control wells. The plates are then incubated for two hours at room temperature with shaking. The plates then are again washed three times. 100 ul of secondary antibody (goat anti-mouse) diluted at 1:5000 in antibody dilution buffer is then added to each well. The secondary antibody is incubated for two hours at room temperature with shaking. An appropriate volume of tetramethyl benzidine (TMB) substrate is aliquoted and kept in the dark. This should be done at the same point in each run to ensure that the TMB reaches a similar temperature prior to use. The plates are then washed three times, then 100 ul of the pre-warmed TMB is added to each well. The plates are then read at 630 nm at a set time point.

### Example 5

### Correlation of Serum JM-27 Levels to BPH

ELISA assays for the level of JM-27 in patient sera were performed, to correlate serum JM-27 levels with BPH. Samples from fifty patients with BPH were tested, along with samples from patients having prostate cancer or other types of cancer. A large majority of samples were run on multiple plates, with good reproducibility.

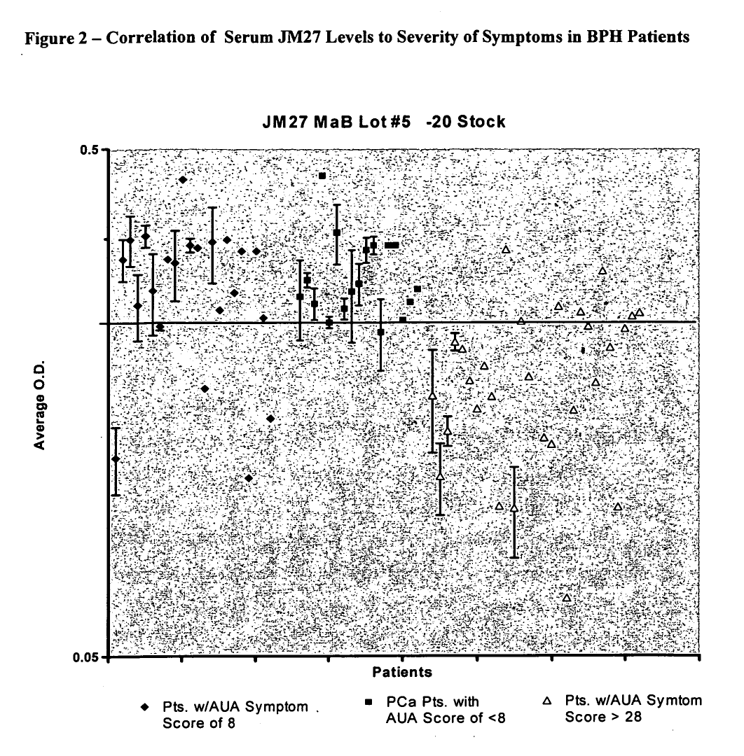

The results revealed a clear discrimination between men with high and low American Urological Association (AUA) symptom scores based solely on serum JM-27 level, and regardless of whether the individuals had prostate cancer. A cutoff was established based on the first run of samples and applied for the remainder of samples. The assay exhibited a sensitivity of 85% and a specificity of 76%. The positive predictive value was 83% and the negative predictive value was 79%. These results indicate that serum JM-27 levels are useful for differentiating individuals with low AUA symptom score BPH (mildly symptomatic) from individuals with high AUA symptom score BPH (severely symptomatic). Surprisingly, BPH patients having higher AUA symptom scores had lower serum JM-27 levels, while patients having lower AUA symptom scores had higher JM-27 levels. See FIGS. 2 & 4, which display the correlation of serum JM-27 levels to the severity of symptoms in BPH patients.

### Example 6

### Sensitivity of the JM-27 ELISA Assay

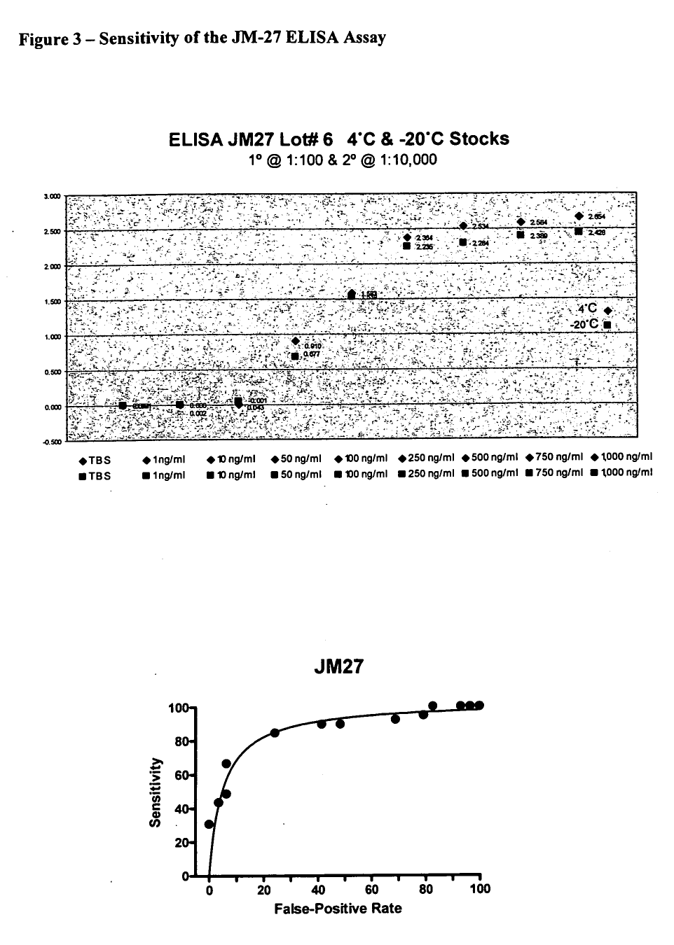

Studies were performed, using standard curves of JM-27 proteins, to demonstrate that the ELISA assay can detect JM-27 in the low ng/ml range. The assay performed in a highly reproducible fashion in these studies, with consistent intra- and inter-assay values. FIG. 3 shows the sensitivity of the ELISA assay using monoclonal antibody stocks stored at both 4° C. and −20° C.

### Example7

### Androgen Regulation of JM-27

The effect of androgen depletion via castration was examined on JM-27 in the rat prostate. The results show that JM-27 expression in both the ventral and dorsolateral lobes is significantly decreased in castrated animals. To verify that the loss of JM-27 expression following castration was indeed an effect attributed to loss of androgens, castrated rats were treated with time-released testosterone pellets (15 mg) immediately following castration and throughout the time period of each study. As expected, exogenous administration of testosterone after castration was able to maintain JM-27 expression in these animals.

1. An assay for detecting JM-27, comprising the steps of (a) providing a serum sample from an individual, and (b) detecting the level of JM-27 in said serum sample.

2. The assay of claim 1, wherein the detection of step (b) employs an anti-JM-27 antibody.

3. The assay of claim 2, wherein said detection of step (b) employs ELISA, immunoprecipitation or western blotting.

4. The assay of claim 2, wherein the detection of step (b) employs a secondary antibody.

5. The assay of claim 4, wherein the detection of step (b) comprises (i) placing said anti-JM-27 antibody in contact with a solid phase coated with said serum, (ii) removing unbound anti-JM-27 antibody; (iii) contacting a secondary antibody with said solid phase, (iv) removing unbound secondary antibody; (v) detecting the level of secondary antibody that is bound to said solid phase; wherein the level of (v) indicates the level of JM-27 in said serum.

6. The assay of claim 5, wherein said secondary antibody is a reporter molecule labeled antibody.

7. The assay of claim 6, wherein said reporter molecule is an enzyme.

8. The assay of claim 2, wherein said anti-JM-27 antibody recognizes and binds to a protein or protein fragment having an amino acid sequence according to SEQ ID NO: 1.

9. A method of diagnosing BPH, comprising (a) providing a serum sample from an individual, (b) detecting the level of JM-27 present in said serum sample, and (c) correlating the detected level of JM-27 to a diagnosis of BPH.

10. The method of claim 9, wherein the detection of step (b) employs an anti-JM-27 antibody.

11. The method of claim 10, wherein said detection of step (b) employs ELISA, immunoprecipitation or western blotting.

12. The method of claim 10, wherein the detection of step (b) employs a secondary antibody.

13. The method of claim 12, wherein the detection of step (b) comprises (i) placing said anti-JM-27 antibody in contact with a solid phase coated with said serum coated, (ii) removing unbound anti-JM-27 antibody; (iii) contacting a secondary antibody with said solid phase, (iv) removing unbound secondary antibody, and (v) detecting the level of secondary antibody that is bound to said solid phase; wherein the level of (v) indicates the level of JM-27 in said serum.

14. The method of claim 12, wherein said secondary antibody is a reporter molecule labeled antibody.

15. The method of claim 14, wherein said reporter molecule is an enzyme.

16. The method of claim 10, wherein said anti-JM-27 antibody recognizes and binds to a protein or protein fragment having an amino acid sequence according to SEQ ID NO: 1.

17. A method of determining the severity of BPH in an individual, comprising (a) providing a serum sample from an individual, (b) detecting the level of JM-27 present in the serum sample, and (c) correlating the detected level of JM-27 to the severity of BPH.

18. A method of monitoring the effectiveness of BPH treatment in an individual, comprising (a) providing a serum sample from an individual, (b) detecting the level of JM-27 present in the serum sample, and (c) comparing the detected level of JM-27 to an earlier serum level of JM-27 in the same individual.

19. The method of claim 18, wherein said treatment comprises the administration of doxazosin to said individual.

20. The method of claim 18, wherein said treatment comprises the administration of finasteride to said individual.
